(19) United States
(12) Patent Application Publication
(10) Pub. No.: US 2007/0270713 A1
(43) Pub. Date: Nov. 22, 2007

(54) Mucous gathering for Pap smear testing

(76) Inventor: Raymond C. Ng, Central (HK)

Correspondence Address: WILLIAM W. HAEFLIGER, 201 S. LAKE AVE, SUITE 512, PASADENA, CA, 91101, US

(21) Appl. No.: 11/436,853

(22) Filed: May 19, 2006

(51) Int. Cl. A61B 10/00

(52) U.S. Cl. 600/570

(57) ABSTRACT

A mucous gathering device, comprising an elongated rotatable stem having an axis, and a rotary head piece carried at an end of the stem for flexing; the headpiece having a face sized for presentation to the cervix entrance as the head rotates about an axis angled relative to the stem; and the face having associated structure for gathering mucous proximate the cervix as the head rotates.

### BACKGROUND OF THE INVENTION

This invention relates generally to cervical cytology examination, and more particularly to improvements in apparatus for conducting such examination.

Cervical cytology examination has well been established as the procedure for the screening of cervical cancer and its precursors since the 1940s. Despite its long history, the procedure for the collection of cervical mucous (with the cervical cells) is far from perfect. Inadequacy rates reported by a study from the UK ranged from 0.2-35.5% (BMJ 1996). Much of the problem has been attributed to failure of collection or sampling devices. The methods as well as devices for the collection of the cervical cells described in this study have remained the same, even though the report was published 10 years ago. There is need for an improved system for the collection of cervical mucous that markedly increases the yield of endo- as well as ecto-cervical cells, for Pap smear cytology examination.

### SUMMARY OF THE INVENTION

It is a major object of the invention to provide improvements in mucous gathering, as referred to above. Basically, the improved cervical mucous gathering device comprises:

a) an elongated rotatable stem having an axis,

b) a rotary head piece carried at an end of the stem for flexing,

c) the headpiece having a face sized for presentation to the cervix entrance as the head rotates about an axis angled relative to the stem,

d) said face having associated means for gathering mucous proximate the cervix as the head rotates.

Additional objects include provision of:

1. A flexible headpiece to facilitate complete contact with the surface of the cervix. Anatomically, the axis of the cervix does not always align with the axis of the vagina. This is due to the mobile positions of the uterus and therefore the cervix. Any uterine tilting changes the plane of the surface of the cervix from a perpendicular position relative to the axis of the vagina. A rigid, flat conventional device makes no allowance for such common anatomical variation, resulting in the incomplete sweeping of the cervical surface. On the contrary, a flexible headpiece as disclosed herein ensures maximum pick-up of the mucous (and cervical cells) during the rotary scraping motion. The flexible headpiece of the device literally “hugs” the cervix throughout the collection process to eliminate non-contact or skip areas. This is especially so when the surface contour is rough and irregular due to physiological or pathological variations. Skip areas account for part of the inadequacy rate of the Pap smear process. The present flexible headpiece is much less traumatic to the cervix as well.

2. The disc or shield type of headpiece as disclosed further ensures complete contact. This is opposite to prior devices, use of which often result in skip areas during rotation. The disc shape allows an even application for a smooth rotary action. Fenestrations on or in the disc plate increase the retention of mucous by the disc itself as by physical capillary action. This headpiece may be detachable for use with liquid preparation methods.

3. A polyester foam wipe may be mounted on the disc plate and is 3-flanged and star-shaped to allow an even and more complete sweep of the surface—a distinct improvement over conventional flat/brush devices. The free edges of the foam wipe are formed to produce an unusually effective mucous gathering effect. The contour is shaped to allow efficient contact of both endo- and ecto-cervial parts of the cervix.

4. The stem piece of the device may be sheathed to allow a smooth rotary action. With one hand of the operator holding the sheath, and at the same time, applying the right amount of gentle pressure against the cervix via the stem for snug and firm contact, the other hand rotates the stem, for example for 1½-2 complete turns, as recommended, to collect the mucous, the axis of the rotating headpiece flexing relative to the axis of the stem, to efficiently follow cervix contour. This two-handed procedure has many advantages as compared to the conventional single-hand method.

A further object is to provide a method of using the said device, which includes the steps
i) manually manipulating said device to bring said face into presentation to the cervix,
ii) manually manipulating the device to rotate the stem and headpiece, while holding the sleeve against rotation, thereby to gather mucous on said face,
   Additional steps may include:
iii) allowing the stem to flex during said step ii),
iv) initially providing flutes on and outstanding from said face, to gather mucous as the face and flutes are rotated to maintain contact with the cervix,
v) forming said flutes to have edges that taper toward an axis of rotation of the face,
vi) providing a rotary head to support said flutes, and manipulating the sleeve to detach the head from the stem following said step ii).

These and other objects and advantages of the invention, as well as the details of an illustrative embodiment, will be more fully understood from the following specification and drawings, in which:

### DRAWING DESCRIPTION

FIG. 1 is a side elevation showing manual manipulation of the device;

FIG. 2 is like FIG. 1 but shows push-pull manipulation of the device, to detach a head;

FIG. 3 is a view showing contact with the cervix and rotation;

FIG. 4 is a view showing a universal connection on the stem, proximate the fluted head; and

FIG. 5 is a perspective view showing details of flutes on the head, and perforations in the head, to gather and retain mucous.

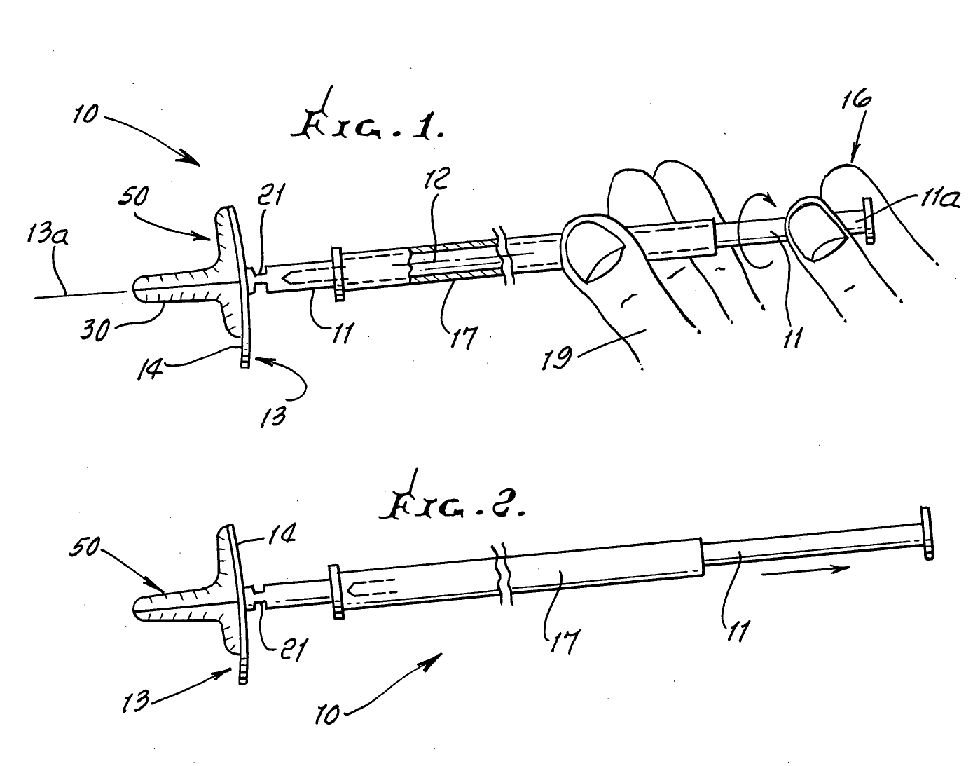

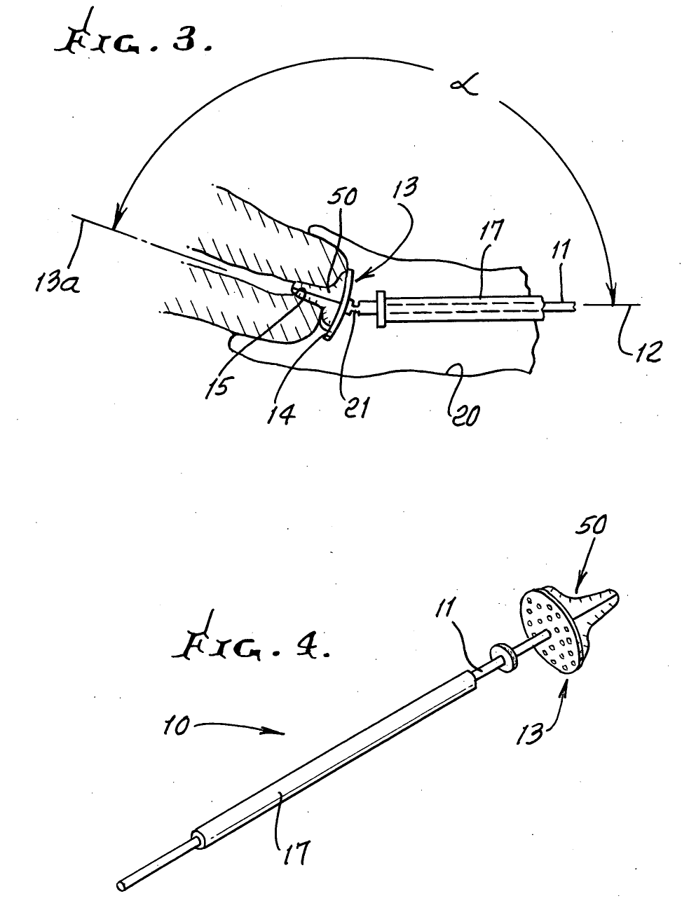

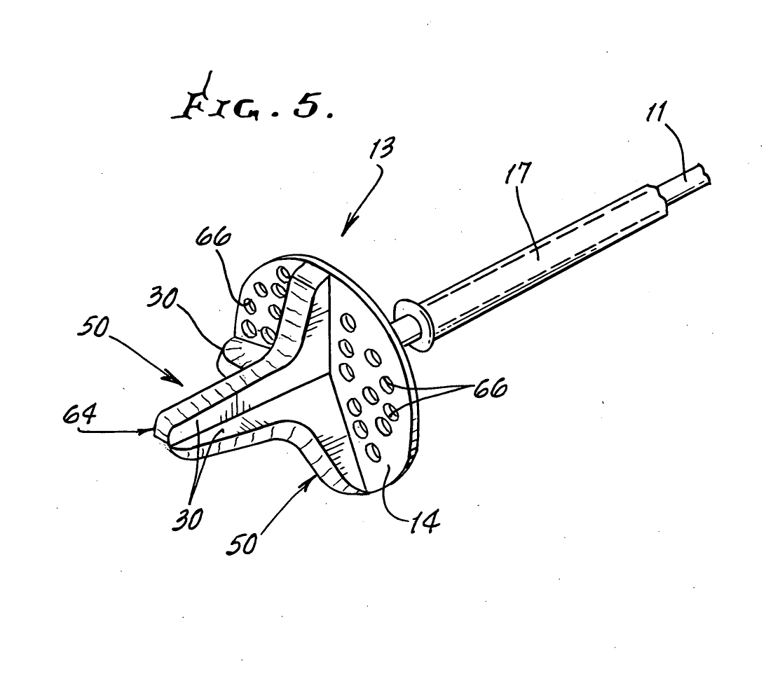

### DETAILED DESCRIPTION

In the drawings, the preferred mucous gather device 10 includes:

a) an elongated rotatable stem 11 having an axis 12 of rotation;

b) a rotary head or head piece 13, shown as circular, carried at the forward end of the stem, for flexing as the head is rotated about its axis 13a, for example at an obtuse angle a relative to axis 12;

c) a face 14 on the head which faces forwardly and is sized for presentation to the cervix entrance 15, as the head is rotated;

d) the face having associated means, such as flutes 50 for gathering mucous proximate the cervix as the head rotates.

Such rotation is typically effected by manual rotation at 16 of a remote end portion 11a of the stem 11. A protective sleeve 17 may extend about the major length of the stem, and is held against rotation by the user's other hand 19, manipulation of the sleeve effecting orientation of the device relative to the vagina 20 and cervix entrance 15. For example, endwise pressure on the sleeve effects pressure transmission to the rotary head as the head rotates.

Such transmission of stem rotation may be transmitted to a universal joint or connection 21 which connects the stem to the head. Connection 21 is adapted to flex to enable head and face rotation about a head axis 13a, when axis 13a may extend at an angle α relative to the stem axis 12, that angle established by head face orientation by the cervix entrance, and stem axis orientation established by the user's manipulation of the sleeve.

Flutes 30 are provided on the head at 120° locations, and may be spaced about axis 13a, and extend on axial radial planes as shown, i.e. in star shape or configuration. The flutes have tapered edges 31, which may be concave in directions away from axis 23, to better fit the entrance configuration of the cervix, as shown in FIG. 5. Corresponding edges may be on a retrievable wipe 64, closely fitting the flute. As the head is rotated, the fluted edges engage and gather mucous and direct flow thereof to the face 14 on the headpiece for collection. The head itself may contain openings or recesses 66 to temporarily store gathered mucous, for later retrieval and pap smear testing or use.

After completion of mucous gathering and withdrawal of the device, the head may be pushed off for detachment from the stem, as by manipulation of the sleeve.

The method of use of the device includes the steps:
i) manually manipulating said device to bring the face into presentation to the cervix,
ii) manually manipulating the device to rotate the stem and headpiece, while holding the sleeve against rotation, thereby to gather mucous on said face, the sleeve manipulated to direct the rotation stem relative to the cervix,
iii) and manually manipulating said device to retrieve said face, and remaining gathered mucous from said face.
   During step ii), the stem extent near the rotating face is allowed to flex, as referred to; also the flutes are provided on and outstanding from the face to gather mucous as the face and flutes are rotated to maintain contact with the cervix. Flutes 30 are shaped to extend away from axis 13a.

I claim:

1. A mucous gathering device, comprising
a) an elongated rotatable stem having an axis,
b) a rotary head piece carried at an end of the stem for angular flexing as the stem rotates,
c) the headpiece having a face sized for presentation to the cervix entrance as the head rotates about an axis angled relative to the stem,
d) said face having associated means for gathering mucous proximate the cervix as the head rotates.

2. The device of claim 1 including a sleeve extending about the stem to be manually manipulated as the stem rotates.

3. The combination of claim 1 wherein said means includes flutes, and disposed about an axis of rotation defined by the face.

4. The combination of claim 3 wherein the flutes have mucous gathering edges that taper toward said face, there being a wipe mounted on said face and forming said edges.

5. The combination of claim 4 wherein said tapered edges are arcuate in directions away from said axis.

6. The combination of claim 4 including perforations in the headpiece to receive mucous gathered in response to flute rotation.

7. The combination of claim 3 including a universal joint located between the stem and head, and connected therewith.

8. The combination of claim 7 wherein the sleeve is sized to fit over said universal joint to exert pressure transmitted to the head.

9. The combination of claim 1 including openings in the headpiece, at its face, to gather and retain mucous, as the head is rotated.

10. The method of using the device of claim 2, which includes
i) manually manipulating said device to bring said face into presentation to the cervix,
ii) manually manipulating the device to rotate the stem and headpiece, while holding the sleeve against rotation, thereby to gather mucous on said face,
iii) and manually manipulating said device to retrieve said headpiece, and remaining gathered mucous.

11. The method of claim 10 including allowing the headpiece to flex relative to the stem, during said step ii).

12. The method of claim 11 including initially providing flutes on and outstanding from said face, to gather mucous as the face and flutes are rotated to maintain flute contact with the cervix.

13. The method of claim 12 including forming said flutes to have smooth edges that taper toward an axis of rotation of the face.

14. The method of claim 12 including manipulating the sleeve to detach the head from the stem following said step ii).

15. The apparatus of claim 1 wherein said headpiece has a universal flex connection to the stem.
